(19) United States
(12) Patent Application Publication
(10) Pub. No.: US 2026/0013482 A1
(43) Pub. Date: Jan. 15, 2026

(54) METHOD FOR CONSTRUCTING AN ANIMAL MODEL OF OPEN-ANGLE GLAUCOMA

(71) Applicant: The Second Xiangya Hospital of Central South University, Changsha (CN)

(72) Inventors: Liu KE, Changsha (CN); Xiaobo WANG, Changsha (CN); Yiwen WANG, Changsha (CN); Rong LIU, Changsha (CN); Xuanchu DUAN, Changsha (CN); Feng ZHANG, Changsha (CN); Jie LV, Changsha (CN); Sihong WU, Changsha (CN)

(73) Assignee: The Second Xiangya Hospital of Central South University, Changsha (CN)

(21) Appl. No.: 19/264,904

(22) Filed: Jul. 10, 2025

(30) Foreign Application Priority Data
Jul. 10, 2024 (CN) 202410923704X

(51) Int. Cl. A01K 67/027; G01N 33/569; G01N 33/58

(52) U.S. Cl. CPC A01K 67/027; G01N 33/56966; G01N 33/582; A01K 2207/10; A01K 2227/105; A01K 2227/107; A01K 2267/03

(57) ABSTRACT

The present disclosure pertains to the field of animal disease models, specifically to a method for constructing an animal model of open-angle glaucoma. In this model, the pathogenesis of open-angle glaucoma is mimicked by inducing necroptosis in trabecular meshwork cells through the injection of IFN-γ into the anterior chamber. This process leads to the inhibition of trabecular meshwork activity and functional impairment, resulting in elevated intraocular pressure and subsequent optic nerve changes indicative of glaucoma. The pathophysiological alterations observed in the animal model closely resemble those seen in human open-angle glaucoma, including chamber angle opening, trabecular meshwork dysfunction, increased intraocular pressure, and loss of RGCs.

### CROSS-REFERENCE TO RELATED APPLICATIONS

The application claims priority to Chinese patent application No. 202410923704X, filed on Jul. 10, 2024, the entire contents of which are incorporated herein by reference.

### TECHNICAL FIELD

The present disclosure pertains to the technical field of animal disease models, specifically describing a method for constructing an animal model of open-angle glaucoma.

### BACKGROUND

Glaucoma is characterized by the progressive and irreversible death of retinal ganglion cells and visual field defects, leading to optic nerve damage and visual function impairment. It is the primary cause of irreversible vision loss worldwide, with an estimated 111.8 million people projected to be affected by 2040. Primary open-angle glaucoma (POAG) is the most common form, accounting for approximately 60%-70% of all glaucoma cases. It is insidious in onset, challenging to diagnose in its early stages, and primarily results from increased aqueous humor outflow resistance. This resistance leads to pathological elevation of intraocular pressure (IOP), causing progressive and irreversible retinal ganglion cell death. Notably, 75% of aqueous humor outflow resistance arises from the trabecular meshwork, underscoring the critical role of maintaining the normal activity and function of trabecular meshwork cells in controlling IOP.

Due to the complex pathogenesis of glaucoma, its molecular pathological mechanisms remain incompletely understood, and studying its pathogenesis directly in humans is highly challenging. Thus, the development of clinically relevant animal models is essential to elucidate the molecular pathology of glaucoma and to devise more effective therapeutic strategies. Advances in glaucoma induction methods, along with improved techniques for quantifying ocular injury, have led to the establishment of various animal models reflecting the pathological and physiological changes observed in human glaucoma. Existing IOP-elevation animal models include anterior chamber injection of microsphere materials, laser photocoagulation, suprascleral vein hypertonic saline injection, suprascleral vein occlusion, transgenic approaches, and steroid-induced models. However, these models present several limitations, including significant disruption of the anatomical structure of aqueous humor circulation, a high incidence of corneal endothelial decompensation, exacerbated inflammatory responses, unstable IOP elevation, and short duration of elevated IOP. Furthermore, most models induce acute IOP elevation, which does not accurately replicate the chronic IOP elevation characteristic of human POAG. Therefore, to investigate the pathological injury mechanisms of POAG and explore novel anti-glaucoma treatments, it is imperative to develop an animal model that more closely mirrors the clinical characteristics of human open-angle glaucoma. In light of these considerations, the present disclosure provides a novel method for constructing an animal model of open-angle glaucoma.

### SUMMARY

The objective of the present disclosure is to provide a method for constructing an animal model of open-angle glaucoma to address the aforementioned challenges.

To achieve this objective, the present disclosure proposes the following technical solution:

A method for constructing an animal model of open-angle glaucoma comprises the following steps:

S1: Selection of specific pathogen-free (SPF) animals as modeling subjects, including C57BL/6J mice (6-8 weeks old), Sprague-Dawley (SD) rats (4-8 weeks old), and Japanese big-eared white rabbits (12-14 months old).

S2: Establishment of an elevated IOP model:

### Anesthesia Procedure:

Animals are weighed prior to anesthesia. General anesthesia is induced in C57BL/6J mice and SD rats via intraperitoneal injection of 1% pentobarbital sodium at a dosage of 40-50 mg/kg, while Japanese big-eared white rabbits receive intramuscular injection of ketamine hydrochloride at a dosage of 22-44 mg/kg. Once deep anesthesia is confirmed, proparacaine hydrochloride eye drops are applied for ocular surface anesthesia.

### IFN-γ Injection Preparation:

IFN-γ stock solutions from different species are diluted with phosphate-buffered saline (PBS) to final concentrations of 9000 Units/ml, 20000 Units/ml, and 30000 Units/ml, respectively.

### Anterior Chamber Injection:

### 1) Mouse/Rat:

(1) The anesthetized mouse/rat is positioned on the operating table of a surgical microscope. A temperature-maintaining mat is placed beneath the table to prevent hypothermia during the procedure. With one eye oriented upward, the eyelid margin is disinfected using a cotton swab dipped in iodophor. Eyelashes are trimmed, and the eyeball is exposed. The ocular surface is irrigated with sterile PBS or ofloxacin antibiotic eye drops, and residual fluid or debris is removed using a dry sterile cotton swab.

(2) The eyeball is stabilized using micro-smooth forceps. A small puncture is created approximately 2 mm outside the pupillary margin using a 34G needle, and a portion of the aqueous humor is released. A microsyringe (10 μl capacity) connected to a 33G needle is inclined at 25° and used to inject IFN-γ (1 μl for mice, 3 μl for rats) through the corneal puncture. Following IFN-γ injection, 2 μl of air is slowly introduced to form a complete bubble in the anterior chamber. The needle remains in position for 30 seconds post-injection to allow the bubble to seal the puncture and prevent backflow. The air bubble is absorbed within a few hours. Care is taken during the injection to avoid damaging the iris and anterior lens capsule. In the experimental group, IFN-γ is injected, while the control group receives an equal volume of PBS.

(3) After the operation, apply ofloxacin antibiotic eye ointment to the eye to prevent infection. Place the mouse on an animal temperature-maintenance device to maintain body temperature, allowing the mouse to recover naturally. Once the mouse has fully recovered, return it to the animal housing room; and

(4) Repeat the anterior chamber injection once a week to maintain the concentration of IFN-γ in the anterior chamber; and

### 2) Rabbit:

Place the anesthetized rabbit directly on a surgical operating table, ensuring that the disinfection procedure is identical to that performed for mice/rats; and

Fix the eyeball using microscopic smooth forceps or a disinfected cotton swab. Make a puncture at the corneoscleral limbus using a 30G needle and release a portion of the aqueous humor. Incline a microsyringe (measuring range: 100 μl) connected to the 30G needle by 25°, and inject IFN-γ (55 μl for rabbits) through the corneal puncture. Maintain the needle in an appropriate position within the anterior chamber for 30 seconds after injection. Slowly withdraw the needle and press the puncture site with a disinfected cotton swab to prevent backflow of the injected reagent. The remaining procedures are identical to those performed for mice/rats; and

### Intraocular Pressure Measurement:

### 1) Mouse/Rat

(1) Gas Inhalation Anesthesia: Prior to each intraocular pressure measurement, anesthetize the mouse/rat using 2%-4% isoflurane mixed with 95% oxygen. Place the mouse/rat in a gas anesthesia machine for 3 to 5 minutes. Confirm the loss of consciousness by squeezing the toes or observing the blinking reflex to assess the anesthetic effect. Apply proparacaine hydrochloride eye drops for ocular surface anesthesia;

(2) Use of TonoLab Rebound Tonometer: Use the TonoLab rebound tonometer, which offers two calibration modes for mice (Π) and rats (r). Align the rebound needle with the center of the cornea and maintain the tonometer in a horizontal position, perpendicular to the corneal surface during measurement. Ensure the mouse/rat is held securely without applying pressure to the neck or orbit, as doing so may artificially elevate intraocular pressure. Measure intraocular pressure using the TonoLab rebound tonometer after inhalation anesthesia. All measurements should be performed by the same operator within the same time period. The TonoLab rebound tonometer automatically calculates a complete measurement value by averaging six consecutive measurements after excluding the highest and lowest values via built-in software. Complete five sets of measurements within two minutes after the mouse/rat loses consciousness and calculate the mean of these five measurements as the final intraocular pressure value; and

(3) Time Points of Intraocular Pressure Measurement: Perform baseline intraocular pressure measurement prior to the initial anterior chamber injection. It is recommended to conduct measurements between 8:00 and 10:00 in the morning. Measure intraocular pressure every other day following anterior chamber injection. Repeat the anterior chamber injection every two weeks during the monitoring period. Monitor intraocular pressure for four weeks, and if the intraocular pressure remains consistently elevated, extend monitoring to every two weeks. The total modeling period lasts for 12 weeks; and

### 2) Rabbit

(1) Measure intraocular pressure in rabbits under topical anesthesia. Secure the rabbit using a rabbit restraining box to facilitate the procedure; and

(2) Use of Tono Vet Rebound Tonometer: Employ the Tono Vet rebound tonometer, which is suitable for rabbits, cats, and dogs, and does not require manual calibration. Align the rebound needle with the center of the rabbit's cornea, keeping the tonometer in a horizontal position and perpendicular to the corneal surface. Ensure that the rabbit's neck is not compressed during fixation, as this may elevate intraocular pressure. The remaining measurement procedures are identical to those used for mice/rats;

S3: Ocular Tissue Examination: At the end of the 4th, 8th, and 12th weeks following the initial anterior chamber injection, euthanize a subset of experimental animals after successful establishment of the elevated intraocular pressure model. Enucleate the eyeball for resin sectioning and hematoxylin-eosin (HE) staining to evaluate the chamber angle opening, trabecular meshwork injury, and retinal damage. Perform fixation, embedding, sectioning (including chamber angle trabecular meshwork and retinal sections), and HE staining on the enucleated eyes. Capture images using an optical microscope to assess structural changes, including the chamber angle opening, trabecular meshwork injury, and retinal damage;

S4: Evaluate glaucomatous optic nerve injury following the successful establishment of the elevated intraocular pressure model.

To evaluate optic nerve injury associated with glaucoma, experimental animals are selected at the end of the 4th, 8th, and 12th weeks following the initial anterior chamber injection. These animals undergo perfusion, and retinal whole mounts are prepared to quantify retinal ganglion cells (RGCs). The extent of RGC loss is assessed to characterize optic nerve damage in the elevated intraocular pressure model and to determine whether the observed injury is consistent with the retinal damage characteristic of glaucoma.

S5: Establishing an intraocular pressure-lowering control group to exclude the influence of elevated intraocular pressure and evaluate retinal injury, thereby verifying the effect of IFN-γ on intraocular pressure, where

IFN-γ is injected into the anterior chamber of the eyes of experimental animals. The anterior chamber is further treated with an intraocular pressure-lowering eye drop. Specifically, 0.5% timolol maleate eye drops are administered twice daily, with one drop applied to the conjunctival sac at 9:00 a.m. and 6:00 p.m. for 12 weeks. The intraocular pressure monitoring method and time points remain consistent with those described above. Intraocular pressure is maintained at baseline levels, or its fluctuation range is comparable to that of the PBS control group for anterior chamber injection, thereby excluding the direct injury of IFN-γ to RGCs. A group of experimental animals is euthanized separately at the end of the 4th, 8th, and 12th weeks, and the eyeballs are removed for resin sectioning and HE staining. Retinal stretching preparation and RGC counting (using the same method described above) are performed to assess whole retinal layer injury and the RGC loss rate. Compared to the IFN-γ anterior chamber injection group without intraocular pressure-lowering drugs, the RGC loss rate is significantly reduced when the intraocular pressure elevation caused by IFN-γ is excluded, and no injury to the whole retinal layer is observed. This confirms the effect of IFN-γ on intraocular pressure.

Preferably, using the mouse as an example, S3 specifically includes the following steps:

### (1) Isolation of the Retina After Mouse Perfusion:

1) Inducing general anesthesia in the mouse by intraperitoneal injection of 1% pentobarbital sodium at a dose of 40 mg/kg-50 mg/kg, securing the mouse on a foam board with needles (inserting the limbs with the needles), and spraying 75% alcohol to moisten the fur.

2) Lifting the skin at the xiphoid process with tweezers and using ophthalmic scissors to cut the skin and ribs of the chest cavity to expose the heart and liver.

3) Clamping the apex of the heart with tweezers, connecting a 10 ml syringe filled with 10 ml of normal saline, carefully inserting the needle into the apex of the heart (ensuring the needle tip does not penetrate too deeply to avoid perfusion into the lungs), and perfusing a small amount of normal saline. The right auricle is then incised using scissors held in the left hand, and the remaining normal saline is perfused until the limbs, liver, and tongue turn white due to blood removal.

4) Keeping the needle fixed in the apex of the heart, removing the syringe, replacing it with a syringe containing 10 ml of 4% paraformaldehyde, and continuing the perfusion. When the paraformaldehyde reaches the brain, a slight reflex phenomenon may be observed in the tail (though this may not always occur). At this point, the perfusion rate is reduced to ensure thorough fixation (fixation mechanism: paraformaldehyde cross-links proteins).

5) Placing the fixed mouse under a microscope and extracting the eyeballs with tweezers, ensuring the eyeballs remain intact.

6) Placing the intact eyeballs in an Eppendorf (EP) tube containing 4% paraformaldehyde and fixing them at room temperature for 1 hour.

7) Puncturing the eyeball with a polypropylene (PP) needle, removing the cornea and iris using trabecular scissors along the puncture site while retaining the lens, and continuing to fix the eyeball in the EP tube containing 4% paraformaldehyde for 2-4hours at room temperature.

8) Removing the lens, carefully separating the choroid and retina by clamping the choroid margin with two tweezers, and fixing the eyeball overnight at 4° C. in 4% paraformaldehyde.

### (2) Retina Stretching Preparation and RGC Staining:

1) Placing the retina in a 24-well plate and washing it with phosphate-buffered saline with Tween 20 (PBST) three times for 15 minutes each (with gentle shaking).

2) Immersing the retina in 50% methanol for 10 minutes (with gentle shaking).

3) Immersing the retina in 100% methanol for 10 minutes (with gentle shaking).

4) Washing the retina again with PBST three times for 15 minutes each (with gentle shaking).

5) Extracting the retina, placing it on a glass slide, cutting it into a four-leaf clover shape with a blade, and spreading it onto the glass slide.

6) Incubating the retina in blocking buffer (10 ml PBS+1% bovine serum albumin (BSA) (0.10 g)+0.5% TritonX-100 (50 μl)) at room temperature for 60 minutes (with gentle shaking).

7) Incubating the retina overnight at 4° C. with a primary antibody targeting ribonucleic acid-binding protein with multiple splicing (RBPMS) under light-protected conditions.

8) Washing the retina with PBST four times for 20 minutes each (with gentle shaking).

9) Incubating the retina with a secondary antibody at room temperature for 2 hours under light-protected conditions.

10) Carefully extracting the retina, placing it on a glass slide, and spreading it to prevent dryness. One drop of PBST is added to each retina. Impurities such as iris fragments are carefully removed using tweezers, and excess liquid is blotted with paper.

11) Applying an anti-fluorescence quenching sealing solution dropwise, covering the retina with a coverslip, sealing the edges with nail polish, allowing the coverslip to dry, and pressing the retina under a tip box containing an appropriate amount of water for 0.5 hours. The prepared retina is stored in a wet box.

### (3) RGCs Counting:

RGCs are photographed and counted using fluorescence microscopy. The mouse retina is divided into four quadrants: dorsal, ventral, nasal, and temporal. Two sites are selected from each quadrant for imaging, and RGCs are counted using ImageJ and ZEN image analysis software. The percentage of RGC loss in the eye subjected to IFN-γ anterior chamber injection is calculated by averaging the counts and comparing the results with the PBS control group.

In this example, during S1, animals are housed under standard conditions with free access to food and water. The experiment commences following a one-week acclimatization period, and all animal care and experimental procedures strictly comply with ethical guidelines for scientific research involving animals.

In this example, during S2, intraocular pressure measurements are as follows:

(1) The baseline intraocular pressure of the mouse prior to anterior chamber injection is 9.5±0.5 mmHg.

(2) The intraocular pressure increases progressively following IFN-γ injection into the anterior chamber:

When IFN-γ (9000 U/mL, administered once weekly) is injected into the anterior chamber of C57BL/6J mice, intraocular pressure exhibits a statistically significant elevation (P<0.05) compared with the control group from the fourth day post-primary injection. The pressure continues to rise steadily, reaching a peak of 30.76±2.55 mmHg approximately 35 days after the initial injection. This elevated intraocular pressure is maintained throughout the observation period (35 to 90 days), representing an ideal model of sustained intraocular pressure elevation.

When IFN-γ (20000 U/mL, administered once weekly) is injected into the anterior chamber of C57BL/6J mice, intraocular pressure increases significantly (P<0.05) from the second day post-primary injection. The pressure peaks at approximately 42 days, reaching 29.53±1.11 mmHg, and remains elevated thereafter, establishing another ideal model of intraocular pressure elevation.

When IFN-γ (30000 U/mL, administered once weekly) is injected into the anterior chamber of C57BL/6J mice, intraocular pressure rises directly to 23.84±0.41 mmHg by the second day post-primary injection. This elevation is statistically significant (P<0.0001) compared with the control group. The pressure remains stably elevated between 2 and 90 days, reaching a maximum of 29.88±1.54 mmHg, representing a robust model of sustained intraocular pressure elevation.

Chamber angle opening and trabecular meshwork injury:

Groups of mice are euthanized at the 4th, 8th, and 12th weeks following the primary anterior chamber injection. Resin sectioning and HE staining are performed on the eyeballs. Optical microscopy reveals that the chamber angle remains open throughout, consistent with the anatomical characteristics of open-angle glaucoma. No significant mechanical injury is observed in the trabecular meshwork.

In this example, during S4, the injury to the entire retinal layer is as follows:

A group of mice were sacrificed at the end of the 4th, 8th, and 12th weeks following the initial anterior chamber injection. Resin sectioning and HE staining were performed on the eyeballs of the mice. Observation under an optical microscope revealed no significant inflammatory injury across the entire retinal layer.

The procedure for retinal ganglion cell (RGC) counting was as follows:

RGCs were specifically labeled through tissue immunofluorescence staining. A group of mice were sacrificed at the end of the 4th, 8th, and 12th weeks following the primary anterior chamber injection of interferon-gamma (IFN-γ). After perfusion, the retinas of both eyes were isolated and spread for RGC counting. The results demonstrated a significant reduction in the number of RGCs in the IFN-γ anterior chamber injection group compared with the control group after 12 weeks of modeling (P<0.05). These findings indicate that anterior chamber injection of IFN-γ effectively induces elevated intraocular pressure in mice and results in glaucomatous optic nerve damage.

In the example, in S2, the direct effects of IFN-γ on the retina following intravitreal injection were evaluated as follows:

IFN-γ and PBS were injected into the vitreous body of experimental animals to assess intraocular pressure changes and retinal injury. Vitreous injection was performed after pupil dilation. Given the large volume of the mouse/rat lens relative to the eye, special attention was paid to the needle's angle to avoid damaging the lens, retina, or other intraocular tissues. To prevent acute elevation of intraocular pressure, an anterior chamber puncture was performed before vitreous injection to release a small amount of aqueous humor. After injection, the needle was kept in place within the vitreous body for approximately 30 seconds before being slowly withdrawn. A conjunctival incision was clamped and closed using microforceps to prevent leakage of the injected solution. The injection cycle lasted for 12 weeks. Groups of experimental animals were sacrificed at the end of the 4th, 8th, and 12th weeks, respectively. Resin sectioning and HE staining (using the previously described method) were performed on the eyeballs to evaluate retinal injury caused by the direct effects of IFN-γ.

Compared with the prior art, the present disclosure has the beneficial effects:

1. Compared with existing methods for establishing an animal model of glaucoma, the present disclosure induces the necroptosis mechanism of trabecular meshwork cells by injecting IFN-γ into the anterior chamber based on the pathogenesis of glaucoma. This induction results in the inhibition of trabecular meshwork activity and impairment of its function, leading to an increase in intraocular pressure and subsequent glaucomatous optic nerve damage. The pathophysiological changes observed in the animal model closely resemble those of human open-angle glaucoma, including an open chamber angle, trabecular meshwork dysfunction, elevated intraocular pressure, and RGC loss, which is positively correlated with the duration of intraocular pressure elevation. This method for glaucoma model preparation is characterized by its innovation, high modeling success rate, short induction period, significant and stable intraocular pressure elevation, and relatively simple operation. Furthermore, the anatomical structure of the normal aqueous humor outflow pathway remains unaltered, making this model highly advantageous and practical.

2. The method for constructing an animal model of open-angle glaucoma simulates the ocular characteristics of human open-angle glaucoma. It establishes an animal model characterized by a short induction period, stable intraocular pressure elevation, and a high modeling success rate. This novel open-angle glaucoma model achieves intraocular pressure elevation without physically blocking the trabecular meshwork or mechanically disrupting the aqueous humor outflow pathway, thereby more accurately reflecting the pathophysiological changes of primary open-angle glaucoma

### BRIEF DESCRIPTION OF THE DRAWINGS

FIG. 1 is a graph showing changes in intraocular pressure following the injection of Mouse IFN-γ (9000 U/ml) or phosphate-buffered saline (PBS) into the anterior chamber of a mouse over a 12-week period.

FIG. 2 is a graph showing changes in intraocular pressure following the injection of Mouse IFN-γ (20000 U/ml) or PBS into the anterior chamber of a mouse over a 12-week period.

FIG. 3 is a graph showing changes in intraocular pressure following the injection of Mouse IFN-γ (30000 U/ml) or PBS into the anterior chamber of a mouse over a 12-week period.

FIG. 4 is a graph illustrating RGC death after the injection of Mouse IFN-γ (9000 U/ml) or PBS into the anterior chamber of a mouse over a 12-week period.

FIG. 5 is a graph illustrating RGC death after the injection of Mouse IFN-γ (20000 U/ml) or PBS into the anterior chamber of a mouse over a 12-week period.

FIG. 6 is a graph illustrating RGC death after the injection of Mouse IFN-γ (30000 U/ml) or PBS into the anterior chamber of a mouse over a 12-week period.

FIG. 7 is a table listing the experimental instruments used in the present disclosure.

FIG. 8 is a table listing the experimental reagents used in the present disclosure.

### DETAILED DESCRIPTIONS OF THE EMBODIMENTS

The technical solutions of the present disclosure will be described clearly and comprehensively below in conjunction with the accompanying figures. The examples provided are intended to illustrate specific embodiments and are not exhaustive. Any other embodiments derived by those skilled in the art based on the examples without requiring inventive efforts fall within the scope of the present disclosure.

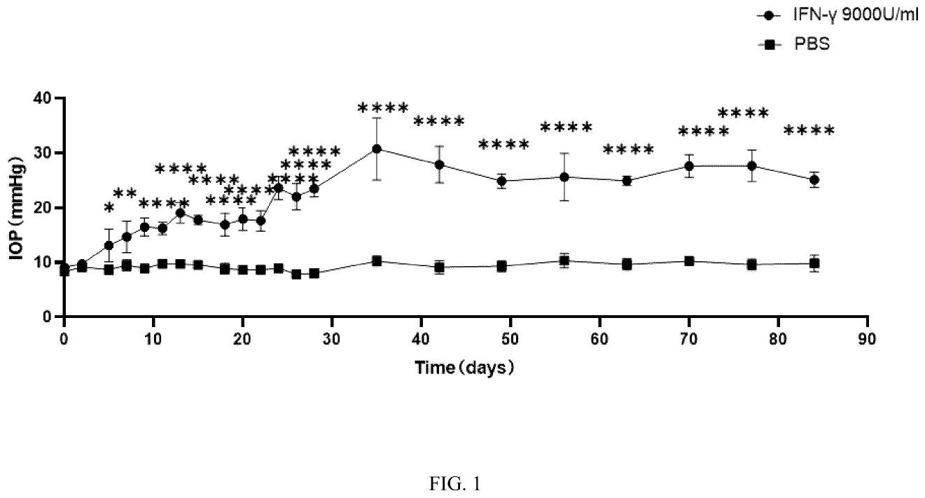

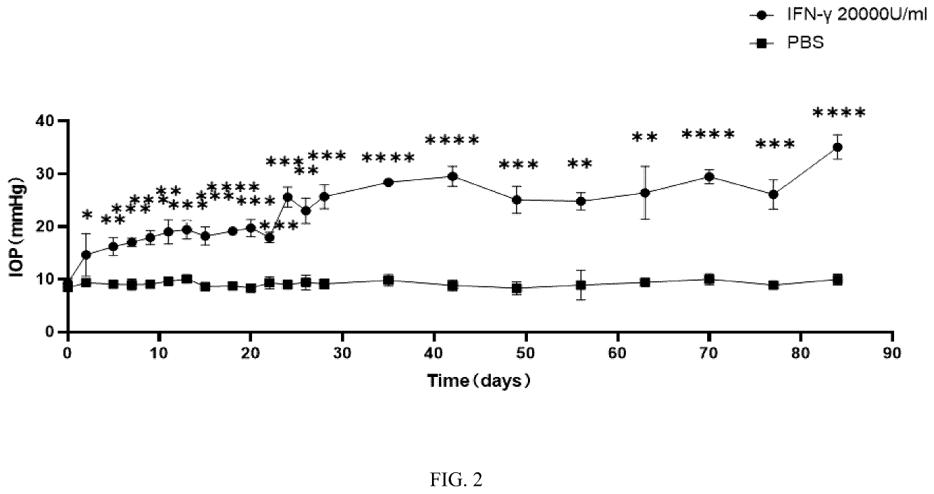

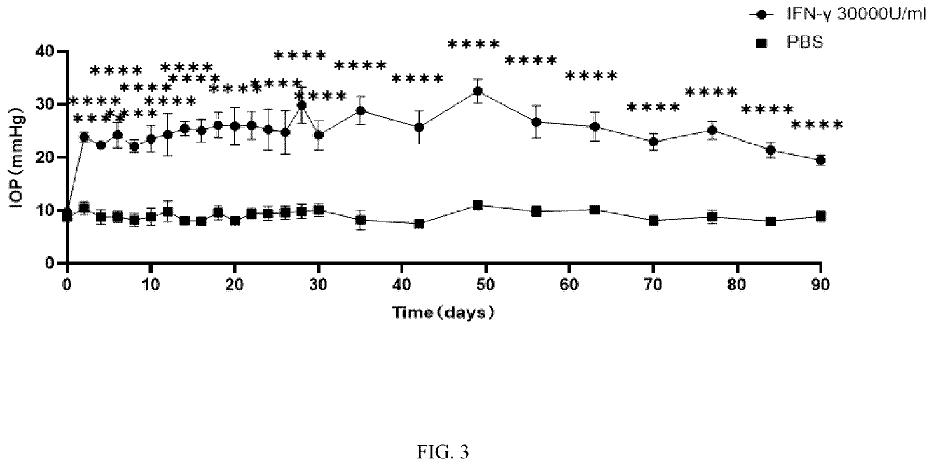

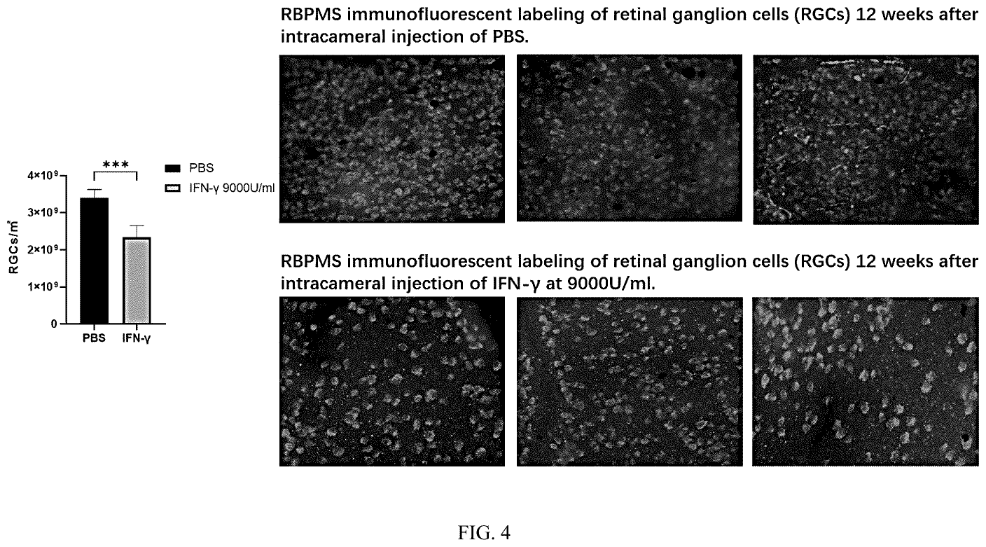

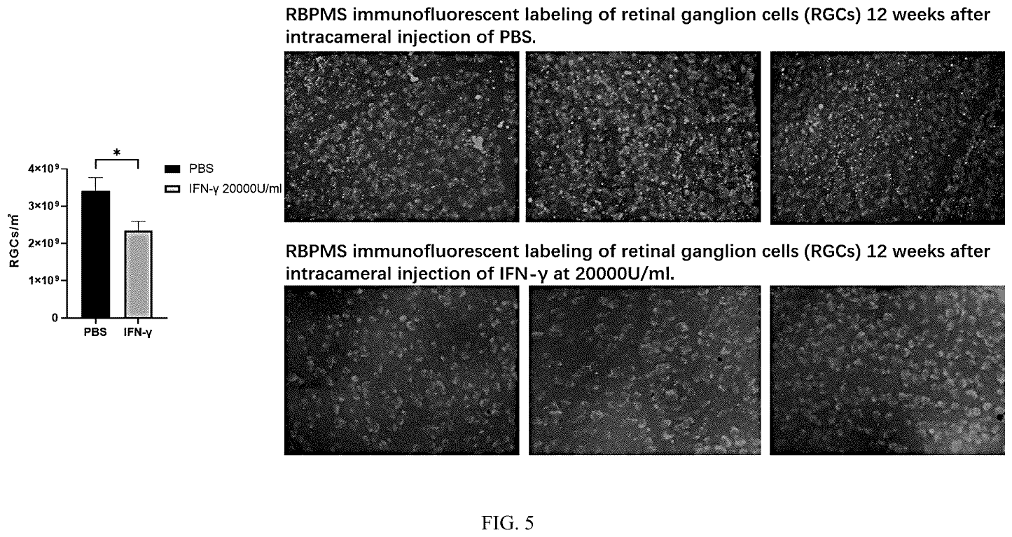

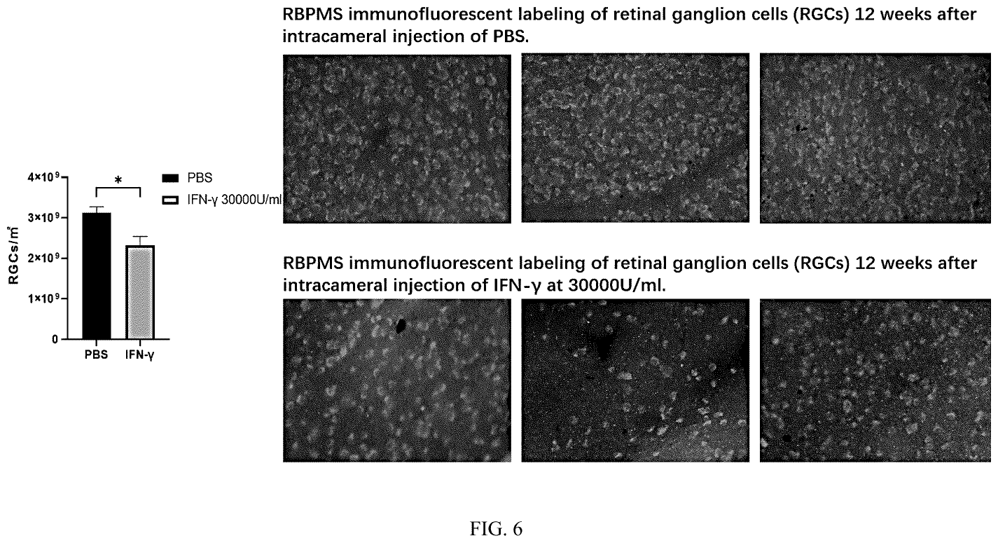

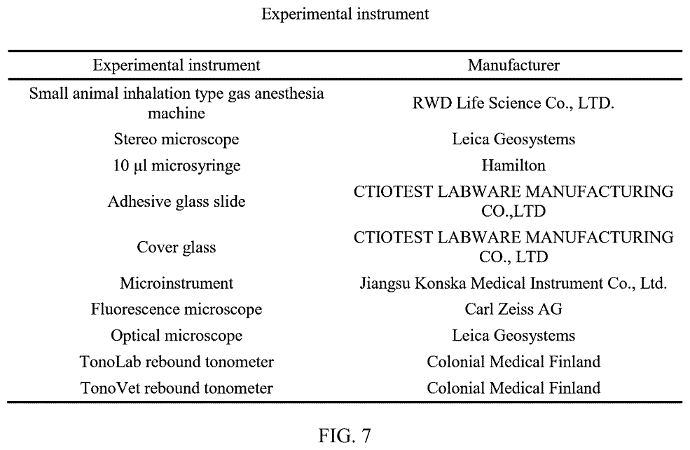

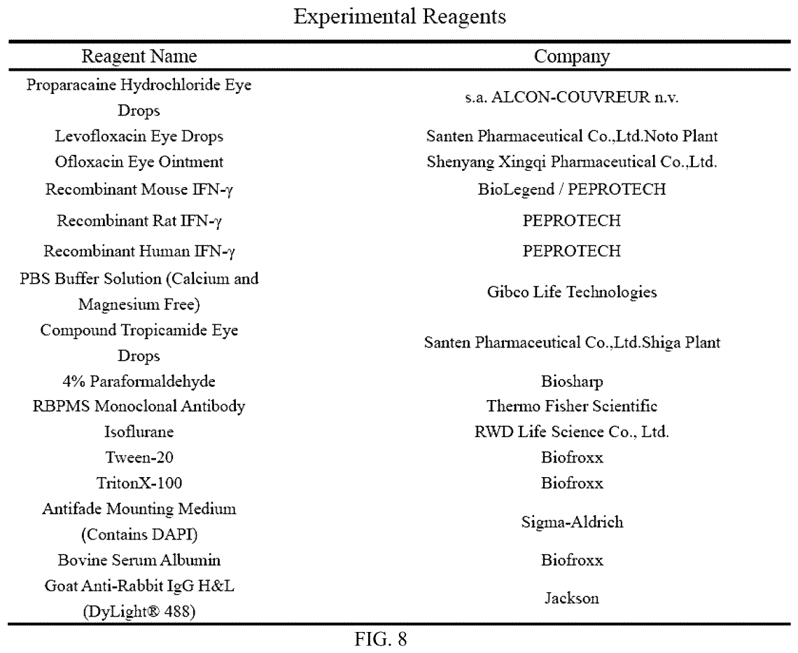

With reference to FIGS. 1-8, the present disclosure provides the following technical solution:

A method for constructing an animal model of open-angle glaucoma comprises the following steps:

S1: Selecting SPF C57BL/6J mice, SD rats, and Japanese big-eared white rabbits as the experimental subjects. The C57BL/6J mice are 6 to 8 weeks old, the SD rats are 4 to 8 weeks old, and the Japanese big-eared white rabbits are 12 to 14 months old.

S2: Establishing the elevated intraocular pressure model:

Anesthesia Method:

The experimental animals are weighed, and general anesthesia is induced as follows: C57BL/6J mice and SD rats receive an intraperitoneal injection of 1% pentobarbital sodium at a dose of 40 mg/kg to 50 mg/kg, while Japanese big-eared white rabbits receive an intramuscular injection of ketamine hydrochloride at a dose of 22 mg/kg to 44 mg/kg. Once deep anesthesia is confirmed, proparacaine hydrochloride eye drops are administered for ocular surface anesthesia.

IFN-γ Injection Preparation:

Mother solutions of IFN-γ from different species are diluted with PBS to final concentrations of 9000 U/ml, 20000 U/ml, and 30000 U/ml.

Anterior Chamber Injection:

### 1) Mouse/rat:

(1) The anesthetized mouse or rat is placed on the operating table of an operating microscope. A heating pad is positioned beneath the animal to maintain body temperature during the procedure. With the eye facing upward, the eyelid margin is disinfected using an iodophor-soaked cotton swab, and the eyelashes are aligned. The ocular surface is rinsed with sterile PBS or ofloxacin antibiotic eye drops, and residual liquid and debris are gently wiped away with a dry, sterile cotton swab.

(2) The eyeball is stabilized using microscopic smooth forceps. A small puncture is made approximately 2 mm from the pupil edge using a 34G needle to release part of the aqueous humor. A microsyringe (10 μl capacity) fitted with a 33G needle is then inclined at 25° to inject IFN-γ (1 μl for mice, 3 μl for rats) through the corneal puncture site. Subsequently, 2 μl of air is slowly injected to form a bubble in the anterior chamber. The needle remains in the anterior chamber for 30 seconds after injection to prevent reflux of the injected solution, after which it is slowly withdrawn. The air bubble acts as a sealant to prevent backflow and is absorbed within a few hours. Care is taken during the puncture process to avoid damaging the iris and anterior lens capsule. The experimental group receives IFN-γ injections, while the control group receives an equal volume of PBS.

(3) After the procedure, ofloxacin antibiotic eye ointment is applied to the eye to prevent infection. The animal is placed on the heating pad for temperature maintenance until full recovery and is subsequently returned to the animal housing facility.

Anterior chamber injections are administered once per week to maintain the concentration of IFN-γ in the anterior chamber; and

### 2) Rabbit

(1) The anesthetized rabbit is placed directly on a surgical operating table, where disinfection preparation is performed in the same manner as for mice and rats; and

(2) The eyeball is stabilized using microscopic smooth forceps or a disinfected cotton swab. A puncture is made at the corneoscleral limbus using a 30G needle to release part of the aqueous humor. Subsequently, a microsyringe (measuring range: 100 μl) connected to the 30G needle is inclined at 25° to inject IFN-γ (55 μl for rabbits) through the corneal puncture. After the injection, the needle remains in the anterior chamber for 30 seconds before being slowly withdrawn. The puncture site is then pressed and sealed using a disinfected cotton swab to prevent backflow of the injection reagent. The remaining procedures are the same as those performed for mice and rats; and

### Intraocular Pressure Measurement:

### 1) Mouse/Rat

(1) Gas Inhalation Anesthesia: Mice and rats are anesthetized using 2%-4% isoflurane mixed with 95% oxygen prior to each intraocular pressure measurement. The animal is placed in a gas anesthesia machine for 3 to 5 minutes. Anesthesia depth is assessed by squeezing the toes or observing the winking reflex to determine whether the animal has lost consciousness. Proparacaine hydrochloride eye drops are administered for ocular surface anesthesia; and

(2) Use of TonoLab Rebound Tonometer: The TonoLab rebound tonometer, which has two calibration modes (mouse: Π, rat: r), is used to measure intraocular pressure. During measurement, the rebound needle is aligned with the center of the cornea while the tonometer is held horizontally and perpendicular to the corneal surface. Care is taken to avoid applying pressure to the neck or orbit, as doing so may artificially elevate intraocular pressure. All intraocular pressure measurements are conducted by the same operator within the same time period. The TonoLab tonometer automatically calculates a final value by removing the highest and lowest readings from six consecutive measurements using built-in software. Five complete measurements are obtained within two minutes after the animal loses consciousness, and the average of these five measurements is recorded as the final intraocular pressure value; and

(3) Time Points for Intraocular Pressure Measurement: Baseline intraocular pressure measurements are performed before the initial anterior chamber injection. It is recommended that measurements be conducted between 8:00 and 10:00 AM. After the anterior chamber injection, intraocular pressure is measured every other day. Injections are repeated every two weeks, and intraocular pressure is monitored for four weeks. If intraocular pressure remains elevated, monitoring continues every two weeks, and the modeling period lasts for 12 weeks; and

### 2) Rabbit

(1) Intraocular pressure in rabbits is measured under topical anesthesia. The rabbit is immobilized using a restraining box to facilitate the procedure; and

(2) Use of TonoVet Rebound Tonometer: The TonoVet rebound tonometer, which is suitable for rabbits, cats, and dogs, and does not require manual calibration, is used for measurement. The rebound needle is aligned with the center of the cornea, and the tonometer is held horizontally and perpendicular to the corneal surface. When using the restraining box, care is taken to avoid compressing the neck, as doing so may elevate intraocular pressure. The remaining measurement procedures are the same as those for mice and rats.

S3: The eyeball is removed to observe the chamber angle opening, trabecular meshwork injury, and retinal injury. Groups of experimental animals are euthanized at the end of the 4th, 8th, and 12th weeks following the initial anterior chamber injection after successful modeling of elevated intraocular pressure. The eyeballs are subsequently removed for resin sectioning and HE staining to evaluate the chamber angle opening, trabecular meshwork injury, and retinal injury. The isolated eyeballs undergo fixation, embedding, sectioning (focusing on the chamber angle trabecular meshwork and retina), and HE staining. The prepared sections are photographed and examined using an optical microscope to assess the chamber angle opening, trabecular meshwork injury, and retinal injury; and

S4: Evaluation of Glaucoma-Related Optic Nerve Injury:

Groups of experimental animals are selected at the end of the 4th, 8th, and 12th weeks following the initial anterior chamber injection. These animals undergo perfusion, followed by retinal wholemount preparation to count RGCs. The loss of RGCs is evaluated to assess optic nerve injury in the elevated intraocular pressure animal model and to determine whether the optic nerve damage is consistent with glaucoma-associated retinal degeneration; and

S5: An intraocular pressure-lowering control group is established to exclude the influence of elevated intraocular pressure and evaluate retinal injury. This allows for the verification of the specific effects of IFN-γ on intraocular pressure.

IFN-γ is injected into the anterior chambers of the eyes of experimental animals. Additionally, the anterior chambers undergo further intervention using an intraocular pressure-lowering eye drop. Specifically, 0.5% timolol maleate eye drops are administered twice daily, with one drop instilled into the conjunctival sac at 9:00 AM and 6:00 PM for 12 weeks. The intraocular pressure monitoring method and time points are consistent with those described in section (3.4). Intraocular pressure is maintained at the baseline level, or its fluctuation range remains comparable to that of the PBS control group receiving anterior chamber injections, thereby excluding the direct impact of IFN-γ on RGCs. Experimental animals are euthanized at the end of the 4th, 8th, and 12th weeks, after which the eyeballs are excised for resin sectioning and HE staining. Retinal stretch preparations and RGC counts (using the previously described method) are performed to assess whole retinal layer injury and the RGC loss rate. Compared to the IFN-γ anterior chamber injection group without intraocular pressure-lowering intervention, the RGC loss rate is significantly reduced when the intraocular pressure elevation caused by IFN-γ is mitigated, and no injury to the whole retinal layer is observed. These findings verify the effect of IFN-γ on intraocular pressure.

In this example, using mice as experimental subjects, S3 specifically includes the following procedures:

### (1) Isolation of the Retina Following Mouse Perfusion:

1) General anesthesia is induced by intraperitoneal injection of 1% pentobarbital sodium at a dose of 40 mg/kg-50 mg/kg. The mouse is secured on a foam board using needles to fix the limbs, and 75% alcohol is sprayed to moisten the fur.

2) The skin at the xiphoid process is lifted using tweezers, and the skin and rib cage are incised with ophthalmic scissors to expose the heart and liver.

3) The apex of the heart is clamped with tweezers. A 10 mL syringe filled with 10 mL of normal saline is carefully inserted into the apex of the heart (ensuring the needle tip does not penetrate too deeply to avoid perfusion into the lungs). A small volume of normal saline is injected, the right atrium is incised with scissors held in the left hand, and the remaining saline is gradually perfused to expel blood. Successful perfusion is indicated by the blanching of the limbs, liver, and tongue.

4) The needle remains fixed in the apex of the heart while the saline-filled syringe is replaced with another syringe containing 10 mL of 4% paraformaldehyde, and perfusion continues. Upon paraformaldehyde reaching the brain, a slight reflex response may be observed in the tail (although not consistently present). At this stage, the perfusion rate is reduced to ensure thorough fixation, as paraformaldehyde induces protein cross-linking.

5) The fixed mouse is positioned under a microscope, and the eyeballs are carefully extracted with tweezers, ensuring they remain intact.

6) The intact eyeballs are placed in EP tubes containing 4% paraformaldehyde and are fixed at room temperature for 1 hour.

7) Using a PP needle, the eyeball is punctured. The cornea and iris are excised along the puncture site with trabecular scissors, leaving the lens intact. The eyeball is then returned to the EP tube with 4% paraformaldehyde and fixed at room temperature for 2-4 hours.

8) The lens is removed, and the choroid margin is clamped using two tweezers. The choroid and retina are carefully separated, and the eyeball is subsequently fixed overnight at 4° C. in 4% paraformaldehyde.

### (2) Preparation of Stretched Retina and RGC Staining:

1) The retina is transferred to a 24-well plate and washed three times with PBST for 15 minutes with gentle shaking.

2) The retina is incubated in 50% methanol for 10 minutes with gentle shaking.

3) The retina is then transferred to 100% methanol for an additional 10 minutes with gentle shaking.

4) The retina is washed three times with PBST for 15 minutes each time, with gentle shaking.

5) The retina is carefully transferred onto a glass slide and cut into a four-leaf clover shape using a blade to facilitate spreading.

6) Blocking buffer (10 mL PBS+1% BSA (0.10 g)+0.5% Triton X-100 (50 μL)) is applied at room temperature for 60 minutes with gentle shaking.

7) The primary antibody specific to RBPMS is incubated overnight at 4° C. under light-protected conditions.

8) The retina is washed four times with PBST for 20 minutes with gentle shaking.

9) The secondary antibody is applied at room temperature for 2 hours under light-protected conditions.

10) The retina is carefully spread onto a glass slide. To prevent drying, a drop of PBST is placed on each retina. Any impurities, such as iris fragments, are meticulously removed using tweezers, and excess liquid is blotted with paper.

11) An anti-fluorescence quenching mounting medium is applied dropwise, and a cover glass is placed over the retina. The edges of the cover glass are sealed with nail polish and allowed to dry. The cover glass is pressed gently with a damp paper in a tip box for 0.5 hours and stored in a humidified chamber.

### (3) RGCs Counting:

RGCs are imaged and quantified using a fluorescence microscope. The retina of each mouse is divided into four quadrants: dorsal, ventral, nasal, and temporal. Two sites from each quadrant are selected for imaging. RGCs in each image are counted using ImageJ and ZEN image analysis software. The percentage of RGC loss in the eyes subjected to anterior chamber injection of IFN-γ is calculated by averaging the RGC counts and comparing them to the PBS control group.

In the example described in S1, the animals are housed in a standard environment with free access to food and water. The experiment is initiated after one week of acclimatization. Animal care and experimental procedures strictly adhere to the ethical guidelines for the use of animals in scientific research.

In the example described in S2, the IOP measurements are as follows:

(1) The baseline IOP of the mice before anterior chamber injection is 9.5±0.5 mmHg.

(2) The IOP gradually increases following IFN-γ injection into the anterior chamber:

When IFN-γ (9000 U/ml, administered once weekly) is injected into the anterior chamber of C57BL/6J mice, the IOP exhibits a statistically significant increase (P<0.05) compared to the control group from the fourth day after the initial injection. The IOP continues to rise steadily and reaches a peak of 30.76±2.55 mmHg approximately 35 days after modeling. The elevated IOP remains stable throughout the observation period (35 to 90 days), establishing a reliable model of elevated intraocular pressure.

When IFN-γ (20000 U/ml, administered once weekly) is injected into the anterior chamber of C57BL/6J mice, a statistically significant increase in IOP (P<0.05) is observed from the second day after the initial injection. The IOP reaches a peak of 29.53±1.11 mmHg approximately 42 days after modeling and remains stable thereafter, forming a reliable model of elevated intraocular pressure.

When IFN-γ (30000 U/ml, administered once weekly) is injected into the anterior chamber of C57BL/6J mice, the IOP rises to 23.84±0.41 mmHg by the second day after the initial injection and exhibits a statistically significant increase compared to the control group (P<0.0001). The IOP remains elevated between days 2 and 90, with a maximum recorded value of 29.88±1.54 mmHg, providing a consistent model of elevated intraocular pressure.

Evaluation of the chamber angle and trabecular meshwork injury:

A cohort of mice is euthanized at the 4th, 8th, and 12th weeks following the initial anterior chamber injection. Resin sectioning and HE staining are performed on the enucleated eyeballs. Observation under an optical microscope reveals that the chamber angle remains open throughout, consistent with the anatomical characteristics of open-angle glaucoma. No apparent mechanical damage is observed in the trabecular meshwork.

In the example described in S4, the evaluation of injury to the entire retinal layer is as follows:

A cohort of mice is euthanized at the 4th, 8th, and 12th weeks following the initial anterior chamber injection. Resin sectioning and HE staining of the enucleated eyeballs reveal no significant inflammatory injury to the entire retinal layer upon microscopic examination.

### Quantification of RGCs:

RGCs are specifically labeled using tissue immunofluorescence staining. At the end of the 4th, 8th, and 12th weeks following anterior chamber injection of IFN-γ, a cohort of mice is euthanized. After perfusion, the retinas from both eyes are isolated and spread for RGC quantification. The results indicate a statistically significant reduction (P<0.05) in the number of RGCs in the IFN-γ injection group compared to the control group after 12 weeks, demonstrating that anterior chamber injection of IFN-γ effectively induces elevated intraocular pressure and causes glaucomatous optic nerve damage.

In the example described in S2, the direct effect of IFN-γ on the retina is evaluated following intravitreal injection:

To assess the direct impact of IFN-γ on the retina, experimental animals receive intravitreal injections of either IFN-γ or PBS. Intraocular pressure and retinal injury are evaluated. Intravitreal injections are performed after pupil dilation. Given the large size of the mouse/rat lens relative to the eye, the needle angle is carefully adjusted to prevent damage to the lens, retina, and other intraocular structures. To avoid acute IOP elevation, anterior chamber puncture is performed prior to intravitreal injection to release a small amount of aqueous humor. Following injection, the needle remains in the vitreous cavity for approximately 30 seconds before being slowly withdrawn. The conjunctival incision is closed using microforceps to prevent leakage. The injection cycle lasts 12 weeks. At the end of the 4th, 8th, and 12th weeks, a cohort of animals is euthanized, and eyeballs are processed for sectioning and HE staining to evaluate retinal injury caused by the direct action of IFN-γ on the retina.

3. According to the method for constructing an animal model of open-angle glaucoma in this example, compared with existing methods for modeling glaucoma, the present disclosure induces a necroptosis mechanism in trabecular meshwork cells by injecting IFN-γ into the anterior chamber. This mechanism leads to the inhibition of trabecular meshwork activity and functional impairment, resulting in elevated intraocular pressure and subsequent glaucomatous optic nerve damage. The pathophysiological changes observed in this animal model closely resemble those of human open-angle glaucoma, including an open chamber angle, trabecular meshwork dysfunction, elevated intraocular pressure, and RGC loss (which is positively correlated with the duration of intraocular pressure elevation). The method for preparing the glaucoma animal model is characterized by innovation, a high success rate of modeling, a short induction period, and a significant and stable elevation of intraocular pressure. The procedure is relatively simple and does not alter the normal anatomical structure of aqueous humor outflow, providing significant advantages and practicality. This method simulates the ocular characteristics of human open-angle glaucoma and establishes an animal model with a short induction period, stable intraocular pressure elevation, and a high modeling success rate. Furthermore, it constructs a novel open-angle glaucoma animal model without elevating intraocular pressure through trabecular meshwork blockage or mechanical damage to the aqueous humor outflow anatomical structure, thereby better reflecting the pathophysiological changes observed in primary open-angle glaucoma.

The foregoing descriptions illustrate the basic principles, essential features, and advantages of the present disclosure. It should be understood by those skilled in the art that the present disclosure is not limited to the above examples. The preferred embodiments described herein are provided for illustrative purposes only and are not intended to limit the scope of the present disclosure. Various changes and modifications may be made without departing from the spirit and scope of the present disclosure, all of which fall within the scope defined by the appended claims and their equivalents.

What is claimed is:

1. A method for constructing an animal model of open-angle glaucoma disease, comprising:
S1: Selecting a SPF C57BL/6J mouse, a SD rat, or a Japanese big-eared white rabbit as the model organism, wherein the C57BL/6J mouse is 6 to 8 weeks old, the SD rat is 4 to 8 weeks old, and the Japanese big-eared white rabbit is 12 to 14 months old;
S2: Establishing an elevated intraocular pressure model:
Anesthesia method:
Weighing the model animals, inducing general anesthesia in the C57BL/6J mouse and SD rat via intraperitoneal injection of 1% pentobarbital sodium at a dose of 40 mg/kg to 50 mg/kg, and inducing general anesthesia in the rabbit via intramuscular injection of ketamine hydrochloride at a dose of 22 mg/kg to 44 mg/kg; Once deep anesthesia is confirmed, proparacaine hydrochloride eye drops are administered for ocular surface anesthesia;
IFN-γ injection preparation: Diluting IFN-γ stock solutions specific to each species with PBS to final concentrations of 9000 Units/ml, 20000 Units/ml, and 30000 Units/ml, respectively;
Anterior chamber injection:
Mouse/rat:
(1) Placing the anesthetized mouse/rat on the operating table of a surgical microscope, with a temperature-controlled mat beneath the table to maintain body temperature during the procedure;
Positioning the eye to be treated upwards, disinfecting the eyelid margin using an iodophor-soaked cotton swab, arranging the eyelashes, and exposing the eyeball; The ocular surface is rinsed with sterile PBS or an ofloxacin antibiotic eye drop, followed by the removal of residual liquid and debris with a sterile dry cotton swab;
(2) The eyeball is stabilized using microscopic smooth forceps, and a small puncture is made approximately 2 mm outside the pupil margin using a 34G needle to release a portion of the aqueous humor; A microsyringe (measuring range: 10 μl) connected to a 33G needle is inclined at an angle of 25° to inject IFN-γ (mouse: 1 μl, rat: 3 μl) through the corneal puncture; Subsequently, 2 μl of air is injected slowly to form a complete bubble in the anterior chamber; The needle of the microsyringe is retained in the anterior chamber for 30 seconds after the injection and then slowly withdrawn, allowing the bubble to seal the corneal puncture and prevent backflow of the injected reagent; The bubble is absorbed within a few hours; Throughout the puncture and injection procedure, care is taken to avoid puncturing the iris or the anterior lens capsule; The experimental group receives IFN-γ as the injection agent, while the control group is administered an equivalent volume of PBS;
(3) Following the procedure, ofloxacin antibiotic eye ointment is applied to prevent infection; The mouse is placed on an animal temperature maintenance device to sustain body temperature during recovery; Once the mouse regains consciousness, it is returned to the animal housing facility;
(4) Anterior chamber injections are repeated once per week to maintain the concentration of IFN-γ in the anterior chamber;
Rabbit:
(1) The anesthetized rabbit is placed directly on a surgical operating table; The disinfection procedure is identical to that of the mouse/rat;
(2) The eyeball is stabilized using microscopic smooth forceps or a sterile cotton swab; A puncture is made at the corneoscleral limbus using a 30G needle to release a portion of the aqueous humor; A microsyringe (measuring range: 100 μl) connected to a 30G needle is inclined at an angle of 25° to inject IFN-γ (rabbit: 55 μl) through the corneal puncture; The needle remains in the anterior chamber for 30 seconds after the injection and is then slowly withdrawn; The puncture site is pressed and sealed with a sterile cotton swab to prevent backflow of the injected reagent; All other procedures are consistent with those for the mouse/rat;
Intraocular Pressure Measurement:
Mouse/Rat:
(1) Gas Inhalation Anesthesia: Prior to each intraocular pressure measurement, the mouse/rat is anesthetized using 2%-4% isoflurane mixed with 95% oxygen; The animal is placed in a gas anesthesia chamber for 3 to 5 minutes; Anesthesia depth is assessed by toe pinch response or the presence of a blinking reflex; Upon confirming anesthesia, a proparacaine hydrochloride eye drop is applied for ocular surface anesthesia;
(2) Use of TonoLab Rebound Tonometer: The TonoLab rebound tonometer, calibrated for both mice and rats (Π=mouse, r=rat), is used to measure intraocular pressure; During measurement, the rebound probe is aligned with the center of the cornea, ensuring the tonometer remains horizontal and perpendicular to the corneal surface; Care is taken to avoid applying pressure to the neck or orbit, as this may artificially elevate intraocular pressure; Measurements are conducted by the same operator within the same time frame; The tonometer automatically calculates a measurement by discarding the highest and lowest values from six consecutive readings; Five complete measurements are performed within two minutes after anesthesia induction, and the mean of these values is recorded as the final intraocular pressure;
(3) Measurement Time Points: Baseline intraocular pressure is measured before the initial anterior chamber injection; Subsequent measurements are taken every other day after injection;
Anterior chamber injections are repeated biweekly, and intraocular pressure is monitored for 4 weeks; If intraocular pressure remains elevated, measurements are performed biweekly for up to 12 weeks;
Rabbit:
(1) Intraocular pressure in rabbits is measured under topical anesthesia; The rabbit is restrained using a rabbit box for ease of handling;
(2) Use of Tono Vet Rebound Tonometer: Intraocular pressure is measured using the Tono Vet rebound tonometer, which is pre-calibrated for rabbits, cats, and dogs and does not require manual adjustment; The probe is aligned with the center of the cornea, and the tonometer is held horizontally and perpendicularly to the corneal surface; Care is taken to avoid compressing the neck when using the rabbit box for restraint, as this can artificially elevate intraocular pressure; Other measurement procedures are identical to those used for mice and rats;
S3: Removal of the Eye for Observation of Chamber Angle Opening, Trabecular Meshwork Injury, and Retinal Injury
A group of experimental animals was euthanized at the end of the 4th, 8th, and 12th weeks following the initial anterior chamber injection, after successful establishment of the elevated intraocular pressure model; The eyeballs were then excised for resin sectioning and HE staining to evaluate the chamber angle opening, trabecular meshwork injury, and retinal injury; The excised eyeballs underwent fixation, embedding, sectioning (primarily including chamber angle trabecular meshwork and retinal sectioning), and HE staining; The prepared sections were photographed and examined under an optical microscope to assess the chamber angle opening, trabecular meshwork injury, and retinal injury;
S4: Evaluation of Glaucoma-Induced Optic Nerve Injury Following the Establishment of the Elevated Intraocular Pressure Model
A group of experimental animals was selected at the end of the 4th, 8th, and 12th weeks following the initial anterior chamber injection for perfusion; The retinas were isolated and prepared for flat-mounting to quantify RGCs; The loss of RGCs was assessed to evaluate optic nerve injury in the elevated intraocular pressure model and to determine whether the observed optic nerve damage corresponded to characteristic glaucomatous retinal degeneration;
S5: Establishment of an Intraocular Pressure-Lowering Control Group to Evaluate the Effect of IFN-γ on Intraocular Pressure
To exclude the influence of elevated intraocular pressure and to verify the direct effect of IFN-γ on retinal injury, an intraocular pressure-lowering control group was established; IFN-γ was injected into the anterior chambers of the experimental animals, followed by intervention using intraocular pressure-lowering eye drops; Specifically, 0.5% timolol maleate eye drops were administered twice daily, with one drop instilled into the conjunctival sac at 9:00 AM and 6:00 PM for 12 weeks; Intraocular pressure was monitored according to the previously described method and time points [3.4]; The intraocular pressure was maintained at baseline levels, or within a fluctuation range comparable to that of the PBS control group receiving anterior chamber injections, to exclude the direct impact of elevated intraocular pressure on RGCs;
A group of experimental animals was euthanized at the end of the 4th, 8th, and 12th weeks, and the eyeballs were removed for resin sectioning, HE staining, retinal flat-mount preparation, and RGC quantification (following the aforementioned methods); The extent of whole retinal layer injury and the RGC loss rate were evaluated and compared with the IFN-γ anterior chamber injection group without intraocular pressure-lowering intervention; The results indicated a significant reduction in the RGC loss rate and an absence of whole retinal layer injury after eliminating the intraocular pressure elevation factor caused by IFN-γ, thereby confirming the specific effect of IFN-γ on intraocular pressure.

2. Method for Constructing an Animal Model of Open-Angle Glaucoma Disease According to claim 1: Mouse Model Procedure
(1) Isolation of the Retina Following Mouse Perfusion:
1) Inducing general anesthesia in the mouse through intraperitoneal injection of 1% pentobarbital sodium at a dose of 40-50 mg/kg; The mouse is then secured onto a foam board using a needle (with the limbs inserted via the needle) and 75% alcohol is sprayed to moisten the fur of the mouse;
2) Lifting the skin at the xiphoid process of the mouse using tweezers, followed by the dissection of the skin and ribs of the chest cavity with ophthalmic scissors to expose the heart and liver;
3) Clamping the apex of the mouse's heart with tweezers, attaching a 10 ml needle to a syringe containing 10 ml of normal saline, and carefully inserting the needle into the apex of the heart (ensuring that the needle tip is not inserted too deeply to avoid perfusion of the lungs); A small volume of normal saline is perfused, followed by the incision of the right auricle using scissors held in the left hand; The remaining saline is then perfused, resulting in the limbs, liver, and tongue turning white;
4) Maintaining the needle in the apex of the heart, the syringe is removed and replaced with a syringe containing 10 ml of 4% paraformaldehyde, and perfusion is continued; When the paraformaldehyde reaches the brain, a slight reflex phenomenon (which may sometimes not be observed) occurs in the mouse's tail; In this case, the perfusion speed is reduced to ensure adequate fixation; The fixation principle involves the cross-linking of proteins by paraformaldehyde;
5) Placing the fixed mouse under a microscope and clamping the eyeball with tweezers, ensuring that the eyeball is not damaged;
6) Transferring the intact eyeball to an EP tube containing 4% paraformaldehyde, and fixing the eyeball for 1 hour at room temperature;
7) Puncturing the eyeball with a PP needle, removing the cornea and iris using trabecular scissors along the ruptured opening, retaining the lens, and continuing fixation of the eyeball in the EP tube containing 4% paraformaldehyde for 2 to 4 hours at room temperature;
8) Removing the lens, clamping the choroid margin with two tweezers, carefully separating the choroid and retina, and continuing fixation of the eyeball in the EP tube containing 4% paraformaldehyde at 4° C. overnight;
(2)) Retinal and RGCs Staining Procedure:
1) Placing the retina in a 24-well plate and washing it with PBST for 15 minutes, repeating three times with gentle shaking;
2) Incubating the retina in 50% methanol for 10 minutes with gentle shaking;
3) Incubating the retina in 100% methanol for 10 minutes with gentle shaking;
4) Washing the retina with PBST for 15 minutes, repeating three times with gentle shaking;
5) Extracting the retina, placing it on a glass slide, and cutting it into a four-leaf clover shape with a blade, spreading the retina on the slide;
6) Incubating the retina in a blocking buffer (10 ml PBS, 1% BSA (0.10 g), 0.5% Triton X-100 (50 μl)) at room temperature for 60 minutes with gentle shaking;
7) Incubating the retina with a primary antibody (targeting RBPMS) overnight at 4° C., protected from light;
8) Washing the retina with PBST for 20 minutes, repeating four times with gentle shaking;
9) Incubating the retina with a secondary antibody at room temperature for 2 hours, ensuring it is kept in the dark;
10) Extracting the retina, placing it on a glass slide, and spreading it carefully; A drop of PBST is added to prevent the retina from drying, and impurities, such as iris fragments, are carefully removed using tweezers in the right hand; Excess liquid is then blotted off with paper;
11) Applying an anti-fluorescence quenching mounting medium dropwise, covering the retina with a cover glass, sealing it with nail polish, and allowing the cover glass to dry; Paper is placed on top of the cover glass, and an appropriate amount of water is added to a tip box, which is then used to press the retina for 30 minutes; The sample is stored in a wet box;
(3) RGCs Counting:
RGCs are photographed and counted using fluorescence microscopy; A retina section from the mouse is divided into four quadrants: dorsal, ventral, nasal, and temporal; Two sites are selected in each quadrant for imaging; The RGCs in the images are counted using ImageJ and ZEN image analysis software; The percentage of RGC loss in the eye following anterior chamber injection of IFN-γ is calculated by averaging the counts and comparing the results with those of the PBS control group.

3. Method for Constructing an Animal Model of Open-Angle Glaucoma (OAG):
In step S1, the animals are housed in a standard environment with free access to water and food; The experiment begins after a week of adaptive feeding, and all procedures are conducted in strict compliance with the ethical guidelines for the use of laboratory animals in scientific research.

4. Intraocular Pressure Measurements in the Animal Model of Open-Angle Glaucoma:
In step S2, the IOP is measured as follows:
(1) The basal IOP of the mouse before the anterior chamber injection is 9.5±0.5 mmHg;
(2)) Following the injection of IFN-γ into the anterior chamber, the IOP gradually increases:
IFN-γ (9000 U/ml, with weekly injections) is administered into the anterior chamber of C57BL/6J mice; The TOP of the mice shows a significant increase compared to the control group from the 4th day after the initial injection (P<0.05), continuing to rise steadily and reaching a peak of 30.76±2.55 mmHg approximately 35 days after modeling; This elevated IOP is maintained at this level during the observation period of 35 to 90 days, representing an ideal elevated IOP model;
IFN-γ (20000 U/ml, with weekly injections) is injected into the anterior chamber of C57BL/6J mice; The IOP shows a significant increase compared to the control group from the 2nd day after the initial injection (P<0.05), reaching a peak of 29.53±1.11 mmHg approximately 42 days post-modeling; The IOP remains stable around this peak throughout the subsequent period, representing an ideal elevated IOP model;
IFN-γ (30000 U/ml, with weekly injections) is injected into the anterior chamber of C57BL/6J mice; The IOP reaches 23.84±0.41 mmHg by the 2nd day after the initial injection and shows a significant increase compared to the control group (P<0.0001); The IOP stabilizes at this elevated level from days 2 to 90, peaking at 29.88±1.54 mmHg, representing an ideal elevated IOP model;
Chamber Angle Opening and Trabecular Meshwork Injury:
A group of mice is euthanized at the 4th, 8th, and 12th weeks after the initial anterior chamber injection; Resin sectioning and HE staining are performed on the eyeballs, and observation under an optical microscope reveals that the chamber angle is open in all quadrants, consistent with the anatomical features of open-angle glaucoma; No significant mechanical injury is observed in the trabecular meshwork.

5. Retinal Injury and RGC Counting:
In step S4, the injury to the entire retinal layer is assessed as follows:
A group of mice is euthanized at the 4th, 8th, and 12th weeks after the initial anterior chamber injection; Resin sectioning and HE staining are performed on the eyeballs, and observation under an optical microscope reveals no significant inflammatory damage to the entire retinal layer;
RGC Counting:
RGCs are specifically labeled through tissue immunofluorescence staining; A group of mice is selected at the 4th, 8th, and 12th weeks following the primary anterior chamber injection of IFN-γ; After perfusion, the retinas of both eyes are dissected, and RGC counts are performed; The results show that the number of RGCs in the IFN-γ-treated group is significantly reduced compared to the control group by the 12th week of modeling (P<0.05); This indicates that the method of anterior chamber injection of IFN-γ effectively induces elevated intraocular pressure in mice and results in optic nerve injury consistent with glaucoma.

6. Method for Constructing an Animal Model of Open-Angle Glaucoma (OAG):
In step S2, following the injection of IFN-γ into the vitreous cavity, the direct effect of IFN-γ on the retina is evaluated;
The experimental groups, consisting of an IFN-γ group and a PBS control group, are administered vitreous injections to assess intraocular pressure and retinal injury; The injection is performed after pupil dilation, and care must be taken due to the large volume occupied by the lens in the mouse/rat eye; Special attention should be given to the angle of the needle tip to avoid damage to the lens, retina, and other intraocular structures; To prevent acute elevation of intraocular pressure, an anterior chamber puncture is performed before vitreous injection to release a small amount of aqueous humor; After the injection, the needle is retained in the vitreous body for approximately 30 seconds before being slowly withdrawn; The conjunctival incision is then clamped and closed using microforceps to prevent leakage of the injected fluid; The injection cycle lasts for 12 weeks, with groups of experimental animals being euthanized at the 4th, 8th, and 12th weeks; Resin sectioning and HE staining are conducted on the eyeballs to evaluate retinal injury caused by the direct action of IFN-γ on the retina.
